(19) United States
(12) Patent Application Publication
(10) Pub. No.: US 2008/0267884 A1
(43) Pub. Date: Oct. 30, 2008

(54) Contrast Agents and Diagnostic Compositions Based on Iodine-Containing Cyanuric Acid Derivatives

(76) Inventors: Oskar Axelsson, Linkoping (SE); Mikkel Thaning, Oslo (NO)

Correspondence Address: GE HEALTHCARE, INC., IP DEPARTMENT, 101 CARNEGIE CENTER, PRINCETON, NJ, 08540-6231, US

(21) Appl. No.: 12/092,871

(22) PCT Filed: Nov. 10, 2005
(86) PCT No.: PCT/NO05/00424

§ 371 Date: May 7, 2008

(51) Int. Cl. C07D 251/34; A61K 49/04

(52) U.S. Cl. 424/9.44; 544/192

(57) ABSTRACT

The present invention relates to a class of compounds of Formula (I) and to diagnostic compositions containing such compounds where the compounds are iodine containing compounds. More specifically the iodine containing compounds are chemical compounds containing a cyanuric acid scaffolding moiety allowing for the arrangement of three iodinated phenyl groups bound thereto. The invention also relates to the use of such diagnostic compositions as contrast agents in diagnostic imaging and in particular in X-ray imaging and to contrast media containing such compounds.

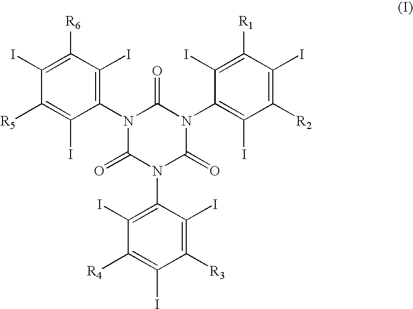

### TECHNICAL FIELD OF THE INVENTION

The present invention relates to a class of compounds and to diagnostic compositions containing such compounds where the compounds are iodine containing compounds. More specifically the iodine containing compounds are chemical compounds containing a cyanuric acid scaffolding moiety allowing for the arrangement of three iodinated phenyl groups bound thereto.

The invention also relates to the use of such diagnostic compositions as contrast agents in diagnostic imaging and in particular in X-ray imaging and to contrast media containing such compounds.

### DESCRIPTION OF RELATED ART

All diagnostic imaging is based on the achievement of different signal levels from different structures within the body. Thus in X-ray imaging for example, for a given body structure to be visible in the image, the X-ray attenuation by that structure must differ from that of the surrounding tissues. The difference in signal between the body structure and its surroundings is frequently termed contrast and much effort has been devoted to means of enhancing contrast in diagnostic imaging since the greater the contrast between a body structure and its surroundings the higher the quality of the images and the greater their value to the physician performing the diagnosis. Moreover, the greater the contrast the smaller the body structures that may be visualized in the imaging procedures, i.e. increased contrast can lead to increased spatial resolution.

The diagnostic quality of images is strongly dependent on the inherent noise level in the imaging procedure, and the ratio of the contrast level to the noise level can thus be seen to represent an effective diagnostic quality factor for diagnostic images.

Achieving improvement in such a diagnostic quality factor has long been and still remains an important goal. In techniques such as X-ray, magnetic resonance imaging (MRI) and ultrasound, one approach to improving the diagnostic quality factor has been to introduce contrast enhancing materials formulated as contrast media into the body region being imaged.

Thus in X-ray early examples of contrast agents were insoluble inorganic barium salts which enhanced X-ray attenuation in the body zones into which they distributed. For the last 50 years the field of X-ray contrast agents has been dominated by soluble iodine containing compounds. Commercial available contrast media containing iodinated contrast agents are usually classified as ionic monomers such as diatrizoate (marketed e.g. under the trade name Gastrografen™), ionic dimers such as ioxaglate (marketed e.g. under the trade name Hexabrix™), nonionic monomers such as iohexyl (marketed e.g. under the trade name Omnipaque™), iopamidol (marketed e.g. under the trade name Isovue™), iomeprol (marketed e.g. under the trade name Iomeron™) and the non-ionic dimer iodixanol (marketed under the trade name and Visipaque™).

The most widely used commercial non-ionic X-ray contrast agents such as those mentioned above are considered safe. Contrast media containing iodinated contrast agents are used in more that 20 millions of X-ray examinations annually in the USA and the number of adverse reactions is considered acceptable. However, since a contrast enhanced X-ray examination will require up to about 200 ml contrast media administered in a total dose, there is a continuous drive to provide improved contrast media.

The utility of the contrast media is governed largely by its toxicity, by its diagnostic efficacy, by adverse effects it may have on the subject to which the contrast medium is administered and by the ease of storage and ease of administration. Since such media are conventionally used for diagnostic purposes rather than to achieve direct therapeutic effect, it is generally desirable to provide media having as little as possible effect on the various biological mechanisms of the cells or the body as this will lead to lower toxicity and lower adverse clinical effect. The toxicity and adverse biological effects of a contrast medium are contributed to by the components of the formulation medium, e.g. the solvent or carrier as well as the contrast agent itself and its components such as ions for the ionic contrast agents and also by its metabolites.

The major contributing factors to the toxicity of the contrast medium are identified as the chemotoxicity of the contrast agent, the osmolality of the contrast medium and the ionic composition or lack thereof of the contrast medium.

Desirable characteristics of an iodinated contrast agent are low toxicity of the compound itself (chemotoxicity), low viscosity of the contrast medium wherein the compound is dissolved, low osmolality of the contrast medium and a high iodine content (frequently measured in g iodine per ml of the formulated contrast medium for administration). The iodinated contrast agent must also be completely soluble in the formulation medium, usually an aqueous medium and remain in solution during storage.

The osmolality of the commercial products, and in particular of the non-ionic compounds is acceptable for most media containing dimers and non-ionic monomers although there is still room for improvement. In coronary angiography for example, injection into the circulatory system of a bolus dose of contrast medium has caused severe side effects. In this procedure contrast medium rather than blood flows through the system for a short period of time, and differences in the chemical and physiochemical nature of the contrast medium and the blood that it replaces can cause undesirable adverse effects such as arrhythmias, QT prolongation and reduction in cardiac contractive force. Such effects are seen in particular with ionic contrast agents where osmotoxic effects are associated with hypertonicity of the injected contrast medium. Contrast media that are isotonic or slightly hypotonic—with the body fluids are particularly desired. Low osmolar contrast media have low renal toxicity which is particularly desirable. The osmolality is a function of the number of particles per volume unit of the formulated contrast medium. To keep the injection volume of the contrast media as low as possible it is highly desirable to formulate contrast media with high concentration of iodine/ml, and still maintain the osmolality of the media at a low level, preferably below or close to isotonicity. The development of non-ionic monomeric contrast agents and in particular non-ionic bis(triiodophenyl) dimers such as iodixanol (EP patent 108638) has provided contrast media with reduced osmotoxicity allowing contrast effective iodine concentration to be achieved with hypotonic solution, and has even allowed correction of ionic imbalance by inclusion of plasma ions while still maintaining the contrast medium Visipaque™ at the desired osmolality (WO 90/01194 and WO 91/13636).

The X-ray contrast media at commercial high iodine concentration have relative high viscosity, ranging from about 15 to about 60 mPas at ambient temperature. Generally, contrast media where the contrast enhancing agent is a dimer has higher viscosity than the corresponding contrast media where the contrast enhancing agent is the monomer corresponding to the dimer. Such high viscosities pose problems to the administrators of the contrast medium, requiring relatively large bore needles or high applied pressure, and are particularly pronounced in pediatric radiography and in radiographic techniques which require rapid bolus administration, e.g. in angiography.

Hence there still exists a desire to develop contrast agents that solves one or more of the problems discussed. Such agents should ideally have improved properties over the soluble iodine containing compounds in one or more of the following properties: renal toxicity, osmolality, viscosity, solubility, injection volumes and attenuation/radiation dose.

### SUMMARY OF THE INVENTION

The present invention provides contrast media having improved properties over the known media with regards to at least one of the following criteria osmolality (and hence the renal toxicity), viscosity and solubility. The contrast media comprises iodine containing contrast enhancing compounds where iodine containing compounds are chemical compounds containing a scaffolding moiety allowing for the arrangement of three iodinated phenyl groups bound to thereto. The iodine containing contrast enhancing compounds can be synthesized from commercially available and relatively inexpensive starting materials.

### DETAILED DESCRIPTION OF THE INVENTION

The contrast enhancing compounds are synthetic chemical compounds of formula (I)

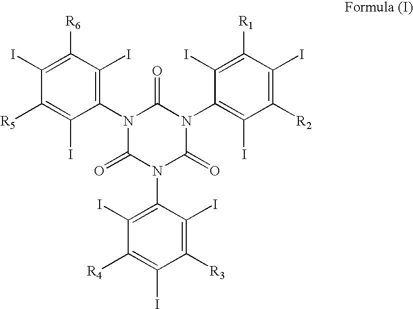

wherein each of the substituents R¹, R², R³, R⁴, R⁵ and R⁶ (hereinafter collectively denoted R group(s)) may be the same or different and denote a hydrogen atom or a non-ionic hydrophilic moiety, provided that at least one R group is a hydrophilic moiety or salts or optical active isomers thereof.

The solubilizing hydrophilic moieties may be any of the non-ionizing groups conventionally used to enhance water solubility. Suitable groups include straight chain or branched chain C₁₋₁₀ alkyl groups, preferably C₁₋₅ alkyl groups, optionally with one or more CH₂ or CH moieties replaced by oxygen or nitrogen atoms and optionally substituted by one or more groups selected from oxo, hydroxyl, amino or carboxyl derivative, and oxo substituted sulphur and phosphorus atoms. Particular examples include polyhydroxyalkyl, hydroxyalkoxyalkyl and hydroxypolyalkoxyalkyl and such groups attached to the phenyl group via an amide linkage such as hydroxyalkylaminocarbonyl, N-alkyl-hydroxyalkylaminocarbonyl and bis-hydroxyalkylaminocarbonyl groups.

In a preferred embodiment the hydrophilic moieties contain 1 to 6 hydroxy groups, preferably 1 to 3 hydroxy groups e.g. groups of the formulas

—CONH—CH₂—CH₂—OH

—CONH—CH₂—CHOH—CH₂—OH

—CONH—CH—(CH₂—OH)₂

—CON—(CH₂—CH₂—OH)₂

—CONH₂

—CONHCH₃

—NHCOCH₂OH

—N(COCH₃)H

—N(COCH₃)C₁₋₃ alkyl

—N(COCH₃)-mono, bis or tris-hydroxy C₁₋₄ alkyl

—N(COCH₂OH)-mono, bis or tris-hydroxy C₁₋₄ alkyl

—N(COCH₂OH)₂

—CON(CH₂—CHOH—CH₂—OH)(CH₂—CH₂—OH)

CONH—C(CH₂—OH)₃ and

CONH—CH(CH₂—OH)(CHOH—CH₂—OH).

Preferably the R groups will be equal or different and denote one or more moieties of the formulas —CONH—CH₂—CHOH—CH₂—OH, —CONH—CH—(CH₂—OH)₂, —CON—(CH₂—CH₂—OH)₂ or —CONH—CH₂—CHOH—CH₂—OH, —NHCOCH₂OH and —N(COCH₂OH)-mono, bis or tris-hydroxy C₁₋₄ alkyl.

Thus examples of preferred structures according to the invention include the compounds of formulas IIa, IIb and IIc below:

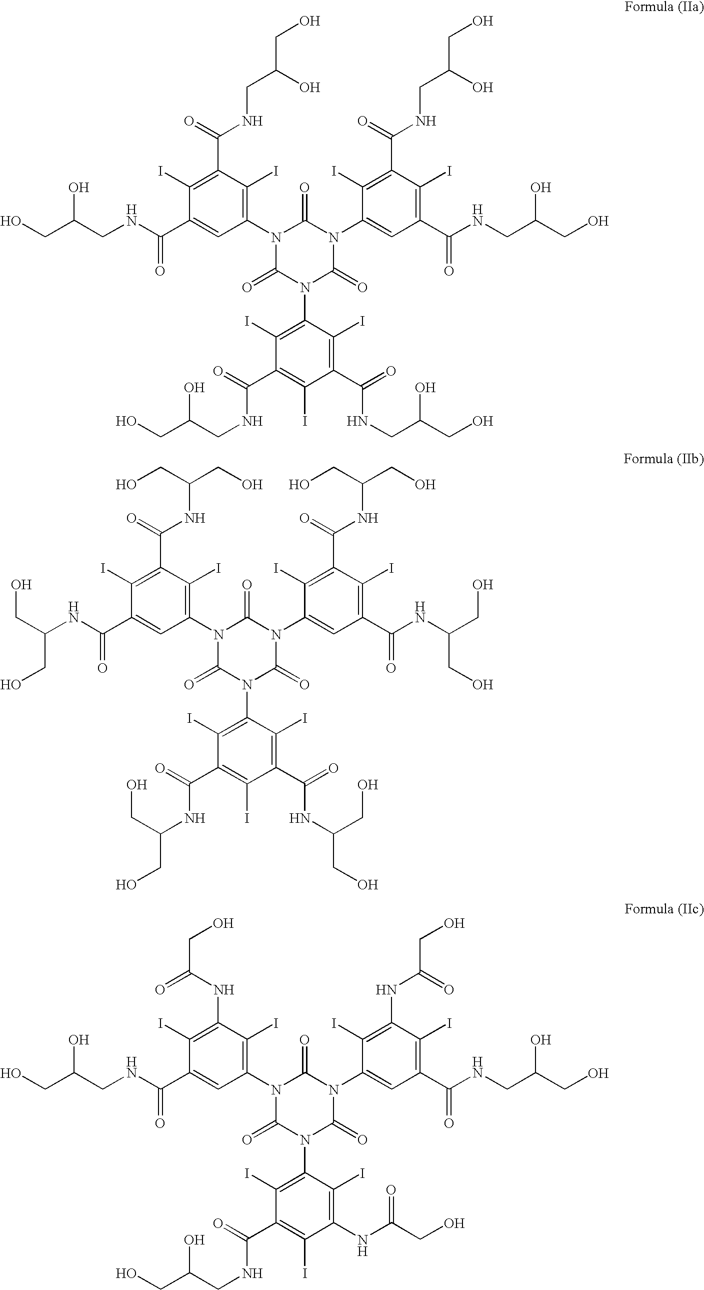

The compounds of formula (I) all have cyanuric acid as the central scaffolding. Cyanuric acid exists in two isomeric forms, the enol and the keto form as shown by Formula (III).

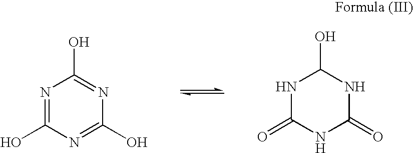

By attaching iodinated phenyl to the cyanuric acid the structure is locked in its keto form. The ortho iodine atoms will force the phenyl groups out of the heterocyclic ring plane, making the molecule adopt a globular form. Globular molecules will have an enhanced solubility compared with molecules with a more planar structure.

The scaffolding heterocyclic cyanuric acid will itself contribute to the solubility of the compound of formula (I) by presenting its polar carboxylic groups to the solvent.

At an iodine concentration of 320 mg/ml which is a common concentration for commercially available iodinated contrast media, the concentration of the compound of formula (I) will be approximately 0.28 M (Molar). The contrast medium will also be hypoosmolar at this iodine concentration, and this is an advantageous property with regards to the nephrotoxicity of the contrast medium. It is also possible to add electrolytes to the contrast medium to lower the cardiovascular effects as explained in WO 90/01194 and WO 91/13636.

Compounds of formula (I) also comprises optical active isomers. Both enantiomerically pure products as well as mixtures of optical isomers are included.

The compounds of the invention may be used as contrast agents and may be formulated with conventional carriers and excipients to produce diagnostic contrast media.

Thus viewed from a further aspect the invention provides a diagnostic composition comprising a compound of formula (I) as described above together with at least one physiologically tolerable carrier or excipient, e.g. in aqueous solution for injection optionally together with added plasma ions or dissolved oxygen.

The contrast agent composition of the invention may be in a ready to use concentration or may be a concentrate form for dilution prior to administration. Generally compositions in a ready to use form will have iodine concentrations of at least 100 mg l/ml, preferably at least 150 mg l/ml, with concentrations of at least 300 mg l/ml, e.g. 320 mg l/ml being preferred. The higher the iodine concentration, the higher is the diagnostic value in the form of X-ray attenuation of the contrast media. However, the higher the iodine concentration the higher is the viscosity and the osmolality of the composition. Normally the maximum iodine concentration for a given contrast media will be determined by the solubility of the contrast enhancing agent, e.g. the iodinated compound, and the tolerable limits for viscosity and osmolality.

For contrast media which are administered by injection or infusion, the desired upper limit for the solution's viscosity at ambient temperature (20° C.) is about 30 mPas, however viscosities of up to 50 to 60 mPas and even more than 60 mPas can be tolerated. For contrast media given by bolus injection, e.g. in angiographic procedures, osmotoxic effects must be considered and preferably the osmolality should be below 1 Osm/kg H₂O, preferably below 850 mOsm/kg H₂O and more preferably about 300 mOsm/kg H₂O.

With the compounds of the invention such viscosity, osmolality and iodine concentrations targets can be met. Indeed, effective iodine concentrations can be reached with hypotonic solutions. It may thus be desirable to make up the solution's tonicity by the addition of plasma cations so as to reduce the toxicity contribution that derives from the imbalance effects following bolus injection. Such cations will desirably be included in the ranges suggested in WO 90/01194 and WO 91/13636.

In particular, addition of sodium and calcium ions to provide a contrast medium isotonic with blood for all iodine concentrations are desirable and obtainable. The plasma cations may be provided in the form of salts with physiologically tolerable counterions, e.g. chloride, sulphate, phosphate, hydrogen carbonate etc., with plasma anions preferably being used.

The compounds of the general formula (I) can be synthesized by several synthetic pathways known to the skilled artisan. Trimerization of isocyanates in the presence of a tertiary amine is one such general pathway followed by periodination and proper functionalization. Isocyanates are available from the reaction of an aniline with phosgene followed by dehydrochlorination. The preparation of cyanuric acid derivatives of formula (I) can be performed according to a scheme which involves the following steps:

a) converting amine(s) of formula (IV)

NH₂—Ar (IV)

wherein Ar denotes a phenyl group substituted by R⁷ at the meta positions with phosgene in toluene to produce isocyanate of formula (V)

O═C═N—Ar (V)

where the R⁷ groups can be the same or different and denote amino groups, nitro groups or carboxylic acid or its derivatives such as esters and amides, followed by
b) dissolving the isocyanate (V) in a polar solvent such as dimethyl sulfoxide and reacting at elevated temperature to form the compound of formula (VI),

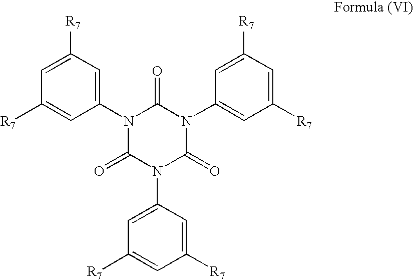

optionally followed by
c) reduction of a nitro containing cyanuric acid derivative using traditional reduction methods, such as catalytic hydrogenation or metal reduction,
followed by
d) iodination of the product using traditional iodination methods to introduce 9 iodine atoms,
followed by
e)
functionalization of amino groups by reaction with optionally protected hydroxylated acid chlorides, such as acetoxyacetyl chloride,
followed by
f) functionalization of carboxylic acid groups into optionally hydroxylated amides using traditional methods and optionally using acid chlorides as intermediates,
followed by
g) optional deprotection of protective groups such as esters and ethers.

The final product is then purified by conventional methods such as preparative HPLC.

Alternatively, the amine(s) if formula (IV) may be triiodinated substituted phenyl groups, in this alternative process the iodination step (d) is omitted.

In step a) the starting amine material (IV) is converted into the corresponding isocyanate (V) by treatment with a solution of phosgene in toluene according to the procedure described in Houben-Weyl: Methoden der Organischen Chemie, Band E4, p. 744, Georg Thieme Verlag, New York 1983. The intermediate isocyanate (V) is then in step b) dissolved in dimethyl sulfoxide at a concentration of about 0.3 M and the solution is heated to about 80° C. After completion of the reaction as determined by analysis of the reaction mixture, the product is isolated by extractive workup followed by purification using either recrystallization or liquid chromatography.

### BRIEF DESCRIPTION OF THE EXAMPLES

The invention will hereinafter be further illustrated with the non-limiting examples. Examples 1 to 5 describes production of compounds of formula (I). All temperatures are in ° C.

The chemical structures of the compounds of formula (I) produced by the examples 1 to 5 below are shown below. The group Ac in formula of Example 2 below depicts an acetyl group.

### EXAMPLES

### Example 1

### N,N′,N″-Tris-[3,5-N,N′-bis-((2,3-dihydroxypronyl)aminocarbonyl)-2,4,6-triiodophenyl]-cyanuric acid

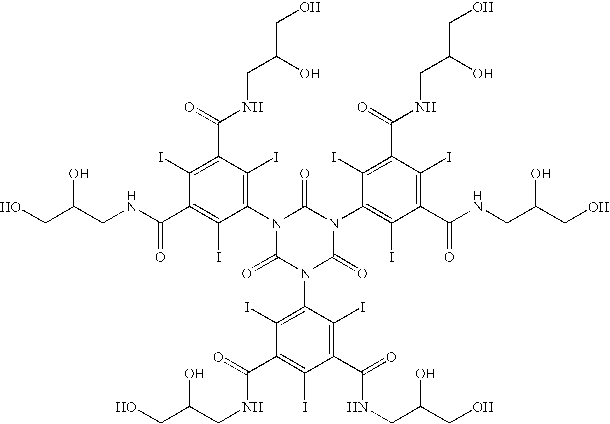

a. 5-Amino-N,N′-bis-(2,3-diacetoxypropyl)-2,4,6-triiodoisophtalamide was synthesized from 5-amino-N,N′-bis-(2,3-dihydroxypropyl)-2,4,6-triiodoisophtalamide via O-acetylation with acetic anhydride in pyridine according to the method described in patent WO 96/09282 (example 1g).

### b. 5-Isocyanato-N,N′-bis-(2,3-diacetoxypropvl)-2,4,6-triiodoisophtalamide

5-Amino-N,N′-bis-(2,3-diacetoxypropyl)-2,4,6-triiodoisophtalamide is dissolved in ethylacetate and treated with a 12 molar excess of phosgene in toluene (1.93 M solution) according to the method in example 3c.

### c. N,N′,N″-Tris-[3,5-N,N′-bis-((2,3-diacetoxypropyl)aminocarbonyl)-2,4,6-triiodophenyl]-cyanuric acid

5-Isocyanato-N,N′-bis-(2,3-diacetoxy propyl)-2,4,6-triiodoisophtalamide is heated in dimethyl sulfoxide according to the procedure in example 3d.

### d. N,N′N″-Tris-[3,5-N,N′-bis-((2,3-dihydroxypropyl)aminocarbonyl)-2,4,6-triiodophenyl]-cyanuric acid

N,N′,N″-Tris-[3,5-N,N′-bis-((2,3-diacetoxypropyl)aminocarbonyl)-2,4,6-triiodophenyl]-cyanuric acid is hydrolyzed with a 15 molar excess of aqueous sodium hydroxide. When the hydrolysis is complete (HPLC-analysis) the mixture is neutralized to pH 5-6 with a strongly acid ion exchange resin (Amberlyst 15). The resin is filtered off and the filtrate is evaporated to dryness. Further purification is performed by HPLC.

### Example 2

### N,N′,N″-Tris-[5-acetamido-3-N-(2,3-dihydroxypropyl)aminocarbonyl-2,4,6-triiodophenyl]-cyanuric acid

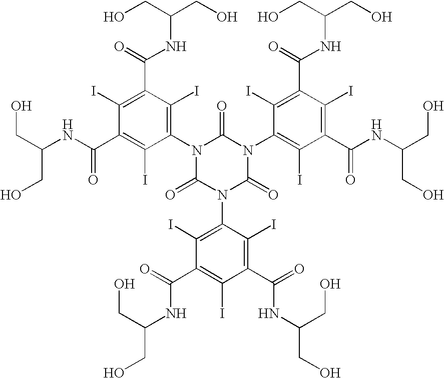

a. 5-Amino-3-acetamido-2,4,6-triiodobenzoic acid was prepared from 3,5-diacetamidobenzoic acid according to the method of U.S. Pat. No. 3,991,105

### b. 5-Amino-3-acetamido-2,4,6-triidodobenzoyl chloride

5-Amino-3-acetamido-2,4,6-triiodobenzoic acid was treated with thionyl chloride in dioxane at 75° C. for 2½ hours. The mixture was then evaporated to dryness, and the residue was redissolved twice in dioxane and evaporated to dryness. The residue was trituated with water for 15 min. and filtered. The light tan coloured product was dried at 40° C. in vacuo (12 torr).

### c. 5-Amino-3-acetamido-N-(2,3-dihydroxypropyl)-2,4,6-triiodobenzamide

5-Amino-3-acetamido-2,4,6-triiodobenzoyl chloride is reacted with two equivalents of 2,3-dihydroxypropylamine in dry tetrahydrofuran for 20 hours at ambient temperature. The salt precipitated after standing over night is filtered off and the filtrate evaporated to a syrup.

### d. 5-Amino-3-acetamido-N-(2,3-diacetoxypropyl)-2,4,6-triiodobenzamide

5-Amino-3-acetamido-N-(2,3-dihydroxypropyl)-2,4,6-triiodobenzamide is O-acetylated according to the method in example 1a.

### e. 5-isocyanato-3-acetamido-N-(2,3-diacetoxypropyl)-2,4,6-triiodobenzamide

5-Amino-3-acetamido-N-(2,3-diacetoxypropyl)-2,4,6-triiodobenzamide in ethyl acetate is treated with phosgene according to the method in example 3c.

### f. N,N′,N″-Tris-[5-acetamido-3-N-(2,3-diacetoxypropyl)aminocarbonyl-2,4,6-triiodophenyl]-cyanuric acid

5-isocyanato-3-acetamido-N-(2,3-diacetoxypropyl)-2,4,6-triiodobenzamide is heated in dimethyl sulfoxide according to the procedure in example 3d.

### g. N,N′,N″-Tris-[5-acetamido-3-N-(2,3-dihydroxypropyl)aminocarbonyl-2,4,6-triiodophenyl]-cyanuric acid

N,N′,N″-Tris-[5-acetamido-3-N-(2,3-diacetoxypropyl)aminocarbonyl-2,4,6-triiodophenyl]-cyanuric acid is hydrolyzed with a 8 molar excess of aqueous sodium hydroxide and worked up according to the method in example 1d.

### Example 3

### N,N′,N″-Tris-[5-hydroxyacetamido-3-N-(2,3-dihydroxypropyl)-carboxamido-2,4,6-triiodophenyl]-cyanuric acid

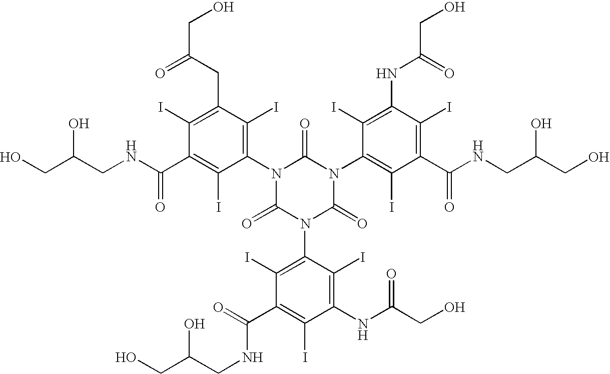

a. 5-Amino-3-nitrobenzoic acid was synthesized from 3,5-dinitrobenzoic acid according to the procedure described in literature. (Larsen et al. J. Am. Chem. Soc. vol. 78, 3210, 1956 or U.S. Pat. No. 3,128,301).

### b. Methyl-5-amino-3-nitrobenzoate

5-Amino-3-nitrobenzoic acid (18.5 g, 0.10 mol) was esterified in methanol (160 ml) by bubbling dry hydrogen chloride into the solution. After saturation, the mixture was stirred over night at ambient temperature. The mixture was then evaporated to a crystalline residue. This was taken up in methylene chloride and washed with diluted sodium hydrogen carbonate solution (5%) until pH 7-8 in aqueous phase. The organic phase was separated, dried (MgSO₄) and the solvent evaporated. Yield: 18.6 g (94%).

¹H NMR (CDCl₃): 8.21 (t, 1H, J=1.5 Hz), 7.63 & 7.61 (2t, 2H, J₁=J₂=1.5 Hz), 4.19 (br. s, 2H), 3.96 (s, 3H).

### c. 5-Nitro-3-carboxymethylphenylisocyanate

Methyl-5-amino-3-nitrobenzoate (5.07 g, 25.9 mmol) was dissolved in ethyl acetate (75 ml). To this solution at ambient temperature was added dropwise a solution of phosgene in toluene (75 ml, 1.93 M) with efficient stirring. The mixture was heated slowly to distil off the solvents. When more than 50% of the solvent mixture was distilled off, the temperature of the residue was decreased to <50° C. Then a new portion of phosgene in toluene (75 ml, 1.93 M) was added and the mixture was again heated slowly to distil off the solvents (110-120° C.). This operation took about 2 h. The last traces of solvents were then distilled off by help of a slight vacuo (200 torr). The resulting oily residue was taken up in dry ether (100 ml), the solution filtered and the solvent evaporated to give a white to yellow crystalline residue. Yield: 5.5 g (96%).

IR: 2256.5 (N═C═O str.), No N—H stretching could be detected.

¹H NMR (CDCl₃): 8.65 (t, 1H, J=1.5 Hz), 8.11 (t, 1H, J=1.5 Hz), 8.08 (t, 1H, J=1.5 Hz), 3.96 (s, 3H).

The product was used directly in next step.

### d. N N′,N″-Tris-[5-nitro-3-carboxymethyl-phenyl]-cyanuric acid

5-Nitro-3-carboxymethylphenylisocyanate (11.2 g, 50.4 mmol) was mixed with dimethyl sulfoxide (10 ml) in a closed flask. The flask was heated to 80° C. for 24 h. After cooling, the contents in the flask were triturated with water (6 ml), filtered and dried. The product was further purified by preparative HPLC. Yield: 10.2 g (91%).

¹H NMR (CD₃COCD₃): 8.87 (t, 3H, J=1.5 Hz), 8.62 (t, 3H, J=1.5 Hz), 8.49 (t, 3H, J=1.5 Hz), 3.98 (s, 9H).

MS (ES⁻, m/e): 701 ([M+Cl⁻]⁻, 14%), 710 ([M+HCOO]⁻, 100%).

### e. N,N′,N″-Tris-[5-nitro-3-carboxy-phenyl]-cyanuric acid

N,N′,N″-Tris-[5-nitro-3-carboxymethyl-phenyl]-cyanuric acid (6.7 g, 10.0 mmol) was suspended in a mixture of dioxane (200 ml) and hydrochloric acid (2 M, 240 ml). The mixture was heated to reflux and held there for 14 h. During this operation a clear colourless solution was left. The solution was then evaporated to dryness and the residue was purified by HPLC. Yield: 6.1 g (97%).

¹H NMR (CD₃COCD₃): 8.88 (t, 3H, J=1.5 Hz), 8.62 (t, 3H, J=1.5 Hz), 8.53 (t, 3H, J=1.5 Hz), 3.58 (br. s, 3H).

MS (ES⁻, m/e): 623 ([M]⁻, 100%).

### f. N,N′,N″-Tris-[5-amino-3-carboxy-2,4,6-triiodophenyl]-cyanuric acid

N,N′,N″-Tris-[5-nitro-3-carboxy-phenyl]-cyanuric acid (2.5 g, 4.1 mmol) was dissolved in a mixture of ethanol (150 ml), water (40 ml) and phosphoric acid (1.0 ml). To this solution was added Pd/C catalyst (10%, 0.6 g) and the solution was hydrogenated at 60 psi in a Parr apparatus. After complete hydrogen consumption the solution was filtered through celite and evaporated to dryness. The product was more than 96% pure according to HPLC analysis and was used without further purification.

MS (ESP⁺, m/e): 534 ([M]⁺, 100%).

The product above was dissolved in water (25 ml). With efficient stirring a water solution (200 ml) of electrochemically generated IBF₄ (see WO 96/09282) in 24 molar excess was added dropwise. The mixture was heated to 60° C. for 96 hours. After cooling to ambient temperature the tan coloured precipitate formed was filtered off washed with a dilute solution of sodium hydrogensulfite (15%, 10 ml), and water (25 ml). The product was purified by preparative HPLC. Yield: 2.7 g (40%).

¹H NMR (DMSO-d₆): 12.65 (br. s, 3H), 3.98 (s, 6H).

¹³C NMR (DMSO-d₆): 170.9, 150.2, 149.3, 146.0, 140.5, 89.9, 89.3, 81.4, 81.0.

MS (ES⁺, m/e): 1668 ([M+H]⁺, 15%), 1541 ([M+H—I]⁺, 100%).

MS (ES⁻, m/e): 1666 ([M−H]⁻, 84%), 1622 ([M-COOH]⁻, 100%).

### g. N N′,N″-Tris-[5-amino-3-chlorocarboxy-2,4,6-triiodophenyl]-cyanuric acid

N,N′,N″-Tris-[5-amino-3-carboxy-2,4,6-triiodophenyl]-cyanuric acid (1.3 g, 0.78 mmol) was suspended in 1,1,1-trichloroethane (8.0 ml). A drop of N,N-dimethyl-formamide was added followed by thionyl chloride (0.90 ml, 11.7 mmol). The mixture was brought to reflux for 6 h, then stirred at ambient temperature over night. The mixture was evaporated, then co-evaporated with 1,1,1-trichloroethane (2×4 ml). The solid residue was trituated with water (5 ml), the precipitate filtered off, washed with water (2 ml) and dried at 40° C. in vacuo (12 torr). Yield: 1.3 g (98%).

¹H NMR (DMSO-d₆): 4.52 (br. s, 6H).

¹³C NMR (DMSO-d₆): 170.3, 170.1, 150.5, 149.9, 148.9, 145.4, 140.0, 80.5, 80.2.

### h. N,N′,N″-Tris-[5-amino-3-N-(3-propenyl)carboxamido-2,4,6-triiodophenyl]-cyanuric acid

N,N′,N″-Tris-[5-amino-3-chlorocarboxy-2,4,6-triiodophenyl]-cyanuric acid (1.26 g, 0.73 mmol) was dissolved in tetrahydrofuran (6 ml) and allylamine (0.49 ml, 6.9 mmol) was added dropwise with efficient stirring. The mixture was stirred at ambient temperature over night, and then evaporated to a solid residue. This was trituated with dilute hydrochloric acid (0.5 M, 4 ml) for 15 min. The precipitate was filtered off, washed with water (2×3 ml) and sucked dry on filter. The product was dried at 30° C. in vacuo (12 torr) to give a tan coloured powder. Yield: 1.26 g (96%).

¹H NMR (DMSO-d₆): 8.61-8.95 (m, 3H), 5.82-6.03 (m, 3H), 5.49 (br. s, 6H), 5.35 (unres. d, 3H), 5.11 (unres. d, 3H), 3.73-3.95 (m, 6H).

¹³C NMR (DMSO-d₆): 170.3, 149.6, 144.2, 139.9, 135.0, 116.7, 116.5, 82.5, 81.5, 42.0.

### i. N,N′,N″-Tris-[5-acetoxyacetamido-3-N-(3-propenyl)carboxamido-2,4,6-triiodophenyl]-cyanuric acid

N,N′,N″-Tris-[5-amino-3-N-(3-propenyl)carboxamido-2,4,6-triiodophenyl]-cyanuric acid (1.24 g, 0.70 mmol) was dissolved in N,N-dimethylacetamide (2.5 ml). At ambient temperature and with efficient stirring, acetoxyacetyl chloride (0.57 g, 4.17 mmol) was added dropwise. Stirring was continued at ambient temperature overnight. The mixture was then poured into a dilute solution of sodium hydrogen carbonate (5%, 10 ml). The tan coloured precipitate formed was filtered off, washed with water (3×5 ml) and sucked dry on filter. The product was dried to a powder at 40° C. in vacuo (12 torr). Yield: 1.25 g (86%).

¹H NMR (DMSO-d₆): 10.11-10.25 (br. s:s, 3H), 8.70-9.12 (m, 3H), 5.81-6.02 (m, 3H), 5.25-5.40 (over). d:s, 3H), 5.04-5.19 (overl. d:s, 3H), 4.64 (s, 6H), 3.77-3.98 (m, 6H), 2.11 (s, 9H).

¹³C NMR (DMSO-d₆): 170.1, 169.4, 165.6, 155.9, 150.9, 145.3, 144.1, 140.8, 134.9, 116.7, 116.5, 86.6, 62.6, 42.0, 21.9, 21.0.

### j. N,N′,N″-Tris-[5-acetoxyacetamido-3-N-(2,3-dihydroxypropyl)carboxamido-2,4,6-triodophenyl]-cyanuric acid

N,N′,N″-Tris-[5-acetoxyacetamido-3-N-(3-propenyl)carboxamido-2,4,6-triiodophenyl]-cyanuric acid (56 mg, 0.027 mmol) was dissolved in a mixture of acetone/water (9/1, 4 ml). Osmium tetroxide (1.5 μmol) was added followed by 4-methylmorpholine N-oxide (20 mg, 0.17 mmol) and the mixture was stirred for 16 h at ambient temperature. A solution of sodium hydrogensulfite (15%, 0.2 ml) was added and the mixture was evaporated to dryness. The product was purified by preparative

HPLC. Yield: 32 mg (54%).

¹H NMR (DMSO-d₆): 10.20-10.28 (s:s, 3H), 8.44-8.92 (m:s, 3H), 4.54-4.80 (m:s+s, 12H), 4.42-4.4.60 (m:s, 3H), 3.60-3.76 & 3.35-3.58 (m:s, 12H), 2.11 (s, 9H).

¹³C NMR (DMSO-d₆): 170.1, 169.7, 165.7, 151.4, 145.3, 144.3, 140.8, 116.3, 107.1, 102.4, 98.5, 70.6, 70.4, 70.2, 64.5, 62.6, 43.1, 21.0.

MS (ES⁺, m/e): 2209 ([M+Na]⁺, 100%), 2226 ([M+K]⁺, 18%).

### k. N,N′,N″-Tris-[5-hydroxyacetamido-3-N-(2,3-dihydroxypropyl)carboxamido-2,4,6-triiodophenyl]-cyanuric acid

N,N′,N″-Tris-[5-acetoxyacetamido-3-N-(2,3-dihydroxypropyl)carboxamido-2,4,6-triiodophenyl]-cyanuric acid (17 mg, 7.8 μmol) was dissolved in a methanol/water mixture (1/4, 1.5 ml) and an aqueous solution of sodium hydroxide (2M, 46 μl) was added at ambient temperature. After stirring for ca. 1 h the mixture was neutralized with a strongly acidic ion exchange resin (Amberlyst 15) to pH 5-6. The resin was filtered off and the aqueous solution was evaporated to dryness. The residue was purified further by preparative HPLC. Yield 12 mg (75%).

MS (ES⁺, m/e): 2010 ([M-3H₂O]⁺, 100%), 2026 ([M-2H₂O]⁺, 27%), 2043 ([M−H₂O]⁺, 4%), 2083 ([M+Na]⁺, 12%).

### Example 4

### N,N′,N″-Tris-[5-hydroxyacetamido-3-N-(2,3-dihydroxypropyl)-carboxamido-2,4,6-triiodophenyl]-cyanuric acid

### a. N,N′,N″-Tris-[5-acetoxacetamido-3-chlorocarboxy-2,4,6-triiodophenyl]-cyanuric acid

N,N′,N″-Tris-[5-amino-3-chlorocarboxy-2,4,6-triiodophenyl]-cyanuric acid prepared in step 3g (0.36 g, 0.21 mmol) was dissolved in dry N,N-dimethylacetamide (2.0 ml), and with efficient stirring at ambient temperature acetoxyacetyl chloride (0.17 ml, 1.52 mmol) was added dropwise. The mixture was stirred over night and the poured into a dilute solution of aqueous sodium hydrogencarbonate (5%, 8.0 ml). The light brown precipitate formed was filtered off, washed with water (2×5 ml), sucked dry on filter and dried at 40° C. in vacuo (12 torr). Yield: 0.40 g (95%).

¹H NMR (DMSO-d₆): 10.31 (br. s, 3H), 4.52 (s, 6H), 2.08 (s, 9H).

¹³C NMR (DMSO-d₆): 170.1, 169.9, 165.7, 157.6, 156.4, 149.7, 145.4, 141.5, 80.2, 61.0, 60.7, 20.8, 20.6.

### b. N,N′,N″-Tris-[5-hydroxyacetamido-3-N-(3-propenyl)carboxamido-2,4,6-triiodophenyl]-cyanuric acid

N,N′,N″-Tris-[5-acetoxyacetamido-3-chlorocarboxy-2,4,6-triiodophenyl]-cyanuric acid (0.69 g, 0.34 mmol) was dissolved in tetrahydrofuran (2.5 ml) at ambient temperature. Allylamine (0.51 ml, 6.8 mmol) was added with efficient stirring. The mixture was stirred for 14 h, and then evaporated to a solid residue. This residue was trituated for 15 min. with a dilute solution of hydrochloric acid (0.05 M, 4.0 ml). The light brown precipitate formed was filtered off, washed with water (2×3 ml) on filter and sucked dry. The filtercake was dried at 40° C. in vacuo (12 torr) to a light brown powder. Yield: 0.65 g (97%).

¹HNMR (DMSO-d₆): 9.75-10.02 (s:s, 3H), 8.59-9.18 (m:s, 3H), 7.28 (s, 3H), 5.44-6.07 (m:s, 6H), 4.81-5.46 (m:s+s:s, 9H), 3.58-4.12 (m:s, 6H).

¹³C NMR (DMSO-d₆): 169.9, 158.9, 156.0, 150.1, 149.6, 145.5, 135.0, 116.5, 82.4, 80.9, 62.3.

### c. N,N′,N″-Tris-[5-hydroxyacetamido-3-N-(2,3-dihydroxypropyl)carboxamido-2,4,6-triiodophenyl]-cyanuric acid

N,N′,N″-Tris-[5-hydroxyacetamido-3-N-(3-propenyl)carboxamido-2,4,6-triiodophenyl]-cyanuric acid (56 mg, 28.6 μmol) was dissolved in an acetone/water mixture (9/1, 6 ml) and treated with osmium tetroxide (1.5 μmol) and 4-methylmorpholine N-oxide (20.0 mg, 172 μmol) according to the conditions in example 3j. The product was purified by preparative HPLC. Yield: 47 mg (79%).

MS (ES⁺, m/2e): 1030 ([M]²⁺, 100%).

1HNMR (DMSO-d₆): 9.84 (s, 3H), 8.42-8.87 (m, 3H), 5.10-6.00 (m, 9H), 4.39-4.96 (m, 9H), 3.40-4.15 (m, 12H).

¹³CNMR (DMSO-d₆): 170.9, 170.4, 166.5, 156.8, 156.0, 151.0, 149.9, 145.5, 140.0, 131.3, 120.3, 116.2, 94.4, 82.5, 70.4, 70.2, 64.8, 64.4, 60.4, 60.1, 43.1, 41.4.

### Example 5

### N,N′,N″-Tris-[5-hydroxyacetamido-3-N-(2,3-dihydroxypropyl)-carboxamido-2,4,6-triiodophenyl]-cyanuric acid

N,N′N″-Tris-[5-acetoxyacetamido-3-chlorocarboxy-2,4,6-triiodophenyl]-cyanuric acid (0.10 g, 49 μmol) obtained in example 4a was dissolved in N,N-dimethylacetamide (1.5 ml). At ambient temperature with efficient stirring 2,3-dihydroxypropylamine (0.08 g, 0.88 mmol) was added. The mixture was stirred for 48 h and then evaporated in high vacuo to a semisolid residue, which was purified by preparative HPLC. Yield: 24 mg (24%).

MS (ES⁺, m/2e): 1030 ([M]²⁺, 31%).

What is claimed is:

1. Compounds of formula (I) 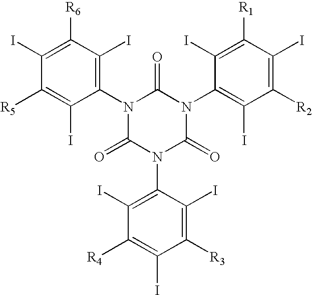
wherein each of the substituents R¹, R², R³, R⁴, R⁵ and R⁶ may be the same or different and denote a hydrogen atom or a non-ionic hydrophilic moiety, provided that at least one R group is a hydrophilic moiety or salts or optical active isomers thereof.

2. Compounds as claimed in claim 1 wherein each of R¹, R², R³, R⁴, R⁵ and R⁶ may be the same or different and are a straight chain or branched chain C₁₋₁₀ alkyl groups optionally with one or more CH₂ or CH moieties replaced by oxygen or nitrogen atoms and optionally substituted by one or more groups selected from oxo, hydroxyl, amino or carboxyl derivative, and oxo substituted sulphur and phosphorus atoms.

3. Compounds as claimed in claim 1 wherein each of R¹, R², R³, R⁴, R⁵ and R⁶ may be the same or different and are a straight chain or branched chain C₁₋₅ alkyl groups optionally with one or more CH₂ or CH moieties replaced by oxygen or nitrogen atoms and optionally substituted by one or more groups selected from oxo, hydroxyl, amino or carboxyl derivative, and oxo substituted sulphur and phosphorus atoms.

4. Compounds as claimed in claim 1 wherein each of R¹, R²R³, R⁴, R⁵ and R⁶ are hydrophilic moieties containing 1 to 6 hydroxy groups, preferably 1 to 3 hydroxy groups.

5. Compounds as claimed in claim 1 wherein each of R¹, R², R³, R⁴, R⁵ and R⁶ are the same or different and are selected from groups of the formulas
—CONH—CH₂—CH₂—OH
—CONH—CH₂—CHOH—CH₂—OH
—CONH—CH—(CH₂—OH)₂
—CON—(CH₂—CH₂—OH)₂
—CONH₂
—CONHCH₃
—NHCOCH₂OH
—N(COCH₃)H
—N(COCH₃)C₁₋₃ alkyl
—N(COCH₃)-mono, bis or tris-hydroxy C₁₋₄ alkyl
—N(COCH₂OH)-mono, bis or tris-hydroxy C₁₋₄ alkyl
—N(COCH₂OH)₂
—CON(CH₂—CHOH—CH₂—OH)(CH₂—CH₂—OH)
—CONH—C(CH₂—OH)₃ and
—CONH—CH(CH₂—OH)(CHOH —CH₂—OH).

6. Compounds as claimed in claim 1 wherein each of R¹, R², R³, R⁴, R⁵ and R⁶ may be the same or different and are polyhydroxyalkyl, hydroxyalkoxyalkyl and hydroxypolyalkoxyalky and such groups attached to the phenyl group via an amide linkage such as hydroxyalkylaminocarbonyl, N-alkyl-hydroxyalkylaminocarbonyl and bis-hydroxyalkylaminocarbonyl groups.

7. Compounds as claimed in claim 1 wherein each of R¹, R², R³, R⁴, R⁵ and R⁶ are the same or different and are selected from groups of the formulas —CONH—CH₂—CHOH—CH₂—OH, —CONH—CH—(CH₂—OH)₂, —CON—(CH₂—CH₂—OH)₂, —CONH—CH₂—CHOH—CH₂—OH, —NHCOCH₂OH and —N(COCH₂OH)-mono, bis or tris-hydroxy C₁₋₄ alkyl.

8. Compound as claimed in claim 1 wherein each of R¹, R², R³, R⁴, R⁵ and R⁶ are equal and are —CONH—CH₂—CHOH—CH₂—OH.

9. Compound as claimed in claim 1 being N,N′,N″-Tris-[5-hydroxyacetamido-3-N-(2,3-dihydroxypropyl)-carboxamido-2,4,6-triiodophenyl]-cyanuric acid.

10. A diagnostic agent comprising a compound of formula (I) as defined in claim 1.

11. A diagnostic composition comprising a compound of formula (I) as defined in claim 1 together with a pharmaceutically acceptable carrier or excipient.

12. An X-ray diagnostic composition comprising a compound of formula (I) as defined in claim 1 together with a pharmaceutically acceptable carrier or excipient.

13-14. (canceled)

15. A method of diagnosis comprising administration of compounds of formula (I) as defined in claim 1 to the human or animal body, examining the body with a diagnostic device and compiling data from the examination.

16. A method of imaging, specifically X-ray imaging, comprising administration of compounds of formula (I) as defined in claim 1 to the human or animal body, examining the body with a diagnostic device and compiling data from the examination and optionally analysing the data.

17. A process for the preparation of a compound of formula (I) as defined in claim 1 comprising the steps of
a) converting amine(s) of formula (IV)
NH₂—Ar (IV)
wherein Ar denotes a phenyl group substituted by R⁷ at the meta positions with phosgene in toluene to produce isocyanate of formula (V)
O═C═N—Ar (V)
where the R⁷ groups can be the same or different and denote amino groups, nitro groups or carboxylic acid or its derivatives such as esters and amides,
b) dissolving the isocyanate (V) in a polar solvent and reacting at elevated temperature to form the compound of formula (VI). 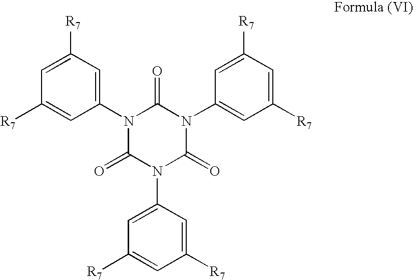
c) optionally reducing a nitro containing cyanuric acid derivative to the corresponding amino compound,
d) iodinating the product from the preceding step by introduction of 9 iodine atoms,
e) functionalizing amino groups by reaction with optionally protected hydroxylated acid chlorides, such as acetoxyacetyl chloride, and/or
f) functionalizing carboxylic acid groups into optionally hydroxylated amides, and
g) optional deprotection of protective groups such as esters and ethers.
